(19) United States
(12) Patent Application Publication
(10) Pub. No.: US 2026/0309706 A1
(43) Pub. Date: Oct. 8, 2026

(54) PORTABLE AND SIMULTANEOUS MASS AND OPTICAL FOOD SAFETY DETECTION TOOL BASED ON SMARTPHONE-BASED QUARTZ CRYSTAL MICROBALANCE

(71) Applicant: Purdue Research Foundation, West Lafayette, IN (US)

(72) Inventors: Joseph Paul ROBINSON, West Lafayette, IN (US); Bartlomiej RAJWA, West Lafayette, IN (US); Euiwon BAE, West Lafayette, IN (US); Jung Joo SOHN, West Lafayette, IN (US); Hyun Jung MIN, West Lafayette, IN (US)

(73) Assignee: Purdue Research Foundation, West Lafayette, IN (US)

(21) Appl. No.: 19/480,735

(22) PCT Filed: Apr. 30, 2024
(86) PCT No.: PCT/US2024/027060

§ 371 Date: Oct. 31, 2025

Related U.S. Application Data
(60) Provisional application No. 63/463,541, filed on May 2, 2023.

(51) Int. Cl. G01N 33/569; G01N 5/00; G01N 21/64; G01N 33/02; G01N 33/58

(52) U.S. Cl. CPC G01N 33/56916; G01N 5/00; G01N 21/645; G01N 33/02; G01N 33/582; G01N 2333/255

(57) ABSTRACT

A method for pathogen detection is also disclosed which includes (i) operating a micropump forcing a first fluid hosting a pathogen-selective antibody from a source to a destination, defining a target, via a microfluidic channel, (ii) operating the micropump to thereby force a second fluid potentially hosting the selective pathogen from the source to the destination, obtaining a signal from the destination indicating a mass change; (iii) comparing the mass change to a predetermined threshold to indicate conditional presence of the pathogen and concentration of the pathogen, (iv) operating the micropump forcing a third fluid hosting a pathogen-selective photoluminescent label from the source to the destination, (v) activating a light excitation subsystem, (vi) capturing images from the target; and (vii) analyzing the images to determine presence of the label thus double-confirming a) presence of the pathogen, and b) concentration of the pathogen.

### CROSS-REFERENCE TO RELATED APPLICATIONS

The present non-provisional patent application is related to and claims the priority benefit of U.S. Provisional Patent Application Ser. 63/463,541, filed May 2, 2023, the contents of which are hereby incorporated by reference in its entirety into the present disclosure.

### STATEMENT REGARDING GOVERNMENT FUNDING

This invention was made with government support under 59-8072-1-002 awarded by the U.S. Department of Agriculture. The government has certain rights in the invention.

### TECHNICAL FIELD

The present disclosure is generally related to pathogen detection and in particular to a system and method based on mass-change measurement and photonic detection when a fluid having a potential pathogen is studied with pathogen-selective antibodies and pathogen-selective photoluminescent label.

### BACKGROUND

This section introduces aspects that may help facilitate a better understanding of the disclosure. Accordingly, these statements are to be read in this light and are not to be understood as admissions about what is or is not prior art.

Foodborne pathogens play havoc in the supply chain of food items every year. These foodborne pathogens often cause acute illnesses when unsuspecting individuals consume tainted food. Typically, these foodborne pathogens are introduced at the farm level or at food processing plants, but they are also possible at any point in the supply chain. The supply chain takes painstaking measures to identify lots and batches. However, because the pathogens are only discovered after the consuming public has already become acutely ill, the solution is reactionary only in the form of destroying vast quantities of food, whether the food is tainted or not. Such reactionary measures are economically disastrous for the companies and the economy.

One of the challenges in detecting microbial pathogens to prevent foodborne illness is a time-sensitive endeavor. Many such detection schemes provide accurate and specific culture-based, molecular-based, immunological, and microscopy-based detection methods. However, these methods are time-consuming, expensive, and require specialized equipment.

Therefore, there is an unmet need for a novel approach to quickly test for the presence of biological pathogens.

### SUMMARY

A pathogen detection system is disclosed. The pathogen detection system includes a microfluidic channel adapted to transfer a volume of fluid from a source to a destination, the destination defining a target. The system further includes a micropump coupled to the microfluidic channel and configured to force fluid along the microfluidic channel from the source to the destination and a mass measurement subsystem adapted to measure changes in mass of a substance at the destination of at least 1 nanogram. The system further includes a light excitation subsystem adapted to project light onto the target, and an image capture subsystem adapted to monitor the target and obtain images therefrom. The system also includes a processing subsystem executing instructions held in a non-transitory memory. The processing subsystem is adapted to: (i) operate the micropump to thereby force a first fluid hosting a pathogen-selective antibody from the source to the destination, (ii) operate the micropump to thereby force a second fluid potentially hosting the selective pathogen from the source to the destination, (iii) obtain a signal from the mass measurement subsystem indicating a mass change between steps (i) and (ii), (iv) compare the mass change to a predetermined threshold to indicate conditional presence of the pathogen and concentration of the pathogen, (v) operate the micropump to thereby force a third fluid hosting a pathogen-selective photoluminescent label from the source to the destination, (vi) activate the light excitation subsystem, capture one or more images from the target by the image capture subsystem, and (vii) analyze the captured one or more images to determine presence of the pathogen-selective photoluminescent label thus double-confirming a) presence of the pathogen, and b) concentration of the pathogen.

In the above system, the mass measurement subsystem includes a quartz crystal microbalance (QCM) adapted to provide a signal corresponding to mass change thereon based on a shift in resonant frequency of the QCM.

In the above system, the selective pathogen is Salmonella Typhimurium, the pathogen-selective antibody is Anti-Salmonella CSA-1, and the pathogen-selective photoluminescent label is FITC-labeled anti-Salmonella CSA-1.

In the above system, the light excitation subsystem is adapted to project light in the range from 250 nm to 900 nm.

In the above system, the pathogen-selective photoluminescent label produces a fluorescence signal.

In the above system, the pathogen-selective photoluminescent label produces a phosphorescence signal.

In the above system, the pathogen-selective photoluminescent label is a small-molecule organic fluorophore.

In the above system, the pathogen-selective photoluminescent label is a nano-particle.

In the above system, the nano-particle is one of a semiconductor-type quantum dot or an up-converting nanoparticle.

In the above system, the image capture subsystem is a smartphone.

In the above system, the image capture subsystem includes a complementary metal oxide semiconductor sensor or a charge-coupled device or a charge-coupled device adapted to capture photoluminescence images.

In the above system, the processing subsystem includes a processor operating the smartphone.

In the above system, volume of the second fluid forced by the micropump is based on a predetermined schedule depending on the selected pathogen.

Another pathogen detection system is also disclosed. The system includes a microfluidic channel adapted to transfer a volume of fluid from a source to a destination, the destination defining a target, a micropump coupled to the microfluidic channel and configured to force fluid along the microfluidic channel from the source to the destination, a mass measurement subsystem adapted to measure changes in mass of a substance at the destination of at least 1 nanogram, a light excitation subsystem adapted to project light onto the target, an image capture subsystem adapted to monitor the target and obtain images therefrom, and a processing subsystem executing instructions held in a non-transitory memory. The processing subsystem is adapted to: (i) operate the micropump to thereby force a first fluid hosting a pathogen-selective antibody from the source to the destination, (ii) operate the micropump to thereby force a second fluid potentially hosting the selective pathogen from the source to the destination, (iii) obtain a first signal from the mass measurement subsystem indicating a first mass change between steps (i) and (ii), (iv) compare the first mass change to a predetermined threshold to indicate conditional presence of the pathogen and concentration of the pathogen, (v) operate the micropump to thereby force a third fluid hosting a pathogen-selective photoluminescent label from the source to the destination, (vi) activate the light excitation subsystem, (vii) capture one or more images from the target by the image capture subsystem, (viii) analyze the captured one or more images to determine presence of the pathogen-selective photoluminescent label thus double-confirming a) presence of the pathogen, and b) concentration of the pathogen, (ix) obtain a second signal from the mass measurement subsystem indicating a second mass change between steps (ii) and (vi), and (x) analyze the second mass change to determine presence of the pathogen-selective photoluminescent label thus triple-confirming a) presence of the pathogen, and b) concentration of the pathogen.

In the above system, the mass measurement subsystem includes a quartz crystal microbalance (QCM) adapted to provide a signal corresponding to mass change thereon based on a shift in resonant frequency of the QCM.

In the above system, the selective pathogen is Salmonella Typhimurium, the pathogen-selective antibody is Anti-Salmonella CSA-1, and the pathogen-selective photoluminescent label is FITC-labeled anti-Salmonella CSA-1.

In the above system, the light excitation subsystem is adapted to project light in the range from 250 nm to 900 nm.

In the above system, the pathogen-selective photoluminescent label produces a fluorescence signal.

In the above system, the pathogen-selective photoluminescent label produces a phosphorescence signal.

In the above system, the pathogen-selective photoluminescent label is a small-molecule organic fluorophore.

In the above system, the pathogen-selective photoluminescent label is a nano-particle.

In the above system, the nano-particle is one of a semiconductor-type quantum dot or an up-converting nanoparticle.

In the above system, the image capture subsystem is a smartphone.

In the above system, the image capture subsystem includes a complementary metal oxide semiconductor sensor or a charge-coupled device or a charge-coupled device adapted to capture photoluminescence images.

In the above system, the processing subsystem includes a processor operating the smartphone.

In the above system, volume of the second fluid forced by the micropump is based on a predetermined schedule depending on the pathogen.

A method of detecting a pathogen is also disclosed. The method includes (i) operating a micropump to thereby force a first fluid hosting a pathogen-selective antibody from a source to a mass measurement subsystem (destination), defining a target, via a microfluidic channel, (ii) operating the micropump to thereby force a second fluid potentially hosting the selective pathogen from the source to the destination, (iii) obtaining a signal from the mass measurement subsystem indicating a mass change between steps (i) and (ii), (iv) comparing the mass change to a predetermined threshold to indicate conditional presence of the pathogen and concentration of the pathogen, (v) operating the micropump to thereby force a third fluid hosting a pathogen-selective photoluminescent label from the source to the destination, (vi) activating a light excitation subsystem, (vii) capturing one or more images from the target by an image capture subsystem, and (vii) analyzing the one or more captured images to determine presence of the pathogen-selective photoluminescent label thus double-confirming a) presence of the pathogen, and b) concentration of the pathogen.

In the above method, the mass measurement subsystem includes a quartz crystal microbalance (QCM) adapted to provide a signal corresponding to mass change thereon based on a shift in resonant frequency of the QCM.

In the above method, the selective pathogen is Salmonella Typhimurium, the pathogen-selective antibody is Anti-Salmonella CSA-1, and the pathogen-selective photoluminescent label is FITC-labeled anti-Salmonella CSA-1.

In the above method, the light excitation subsystem is adapted to project light in the range from 250 nm to 900 nm.

In the above method, the pathogen-selective photoluminescent label produces a fluorescence signal.

In the above method, the pathogen-selective photoluminescent label produces a phosphorescence signal.

In the above method, the pathogen-selective photoluminescent label is a small-molecule organic fluorophore.

In the above method, wherein the pathogen-selective photoluminescent label is a nano-particle.

In the above method, the nano-particle is one of a semiconductor-type quantum dot or an up-converting nanoparticle.

In the above method, the image capture subsystem is a smartphone.

In the above method, the image capture subsystem includes a complementary metal oxide semiconductor sensor or a charge-coupled device or a charge-coupled device adapted to capture photoluminescence images.

In the above method, the processing subsystem includes a processor operating the smartphone.

In the above method, volume of the second fluid forced by the micropump is based on a predetermined schedule depending on the pathogen.

Another method of detecting a pathogen is also disclosed. The method includes (i) operating a micropump to thereby force a first fluid hosting a pathogen-selective antibody from a source to a mass measurement subsystem (destination), defining a target, via a microfluidic channel, (ii) operating the micropump to thereby force a second fluid potentially hosting the selective pathogen from the source to the destination, (iii) obtaining a signal from the mass measurement subsystem indicating a mass change between steps (i) and (ii), (iv) comparing the mass change to a predetermined threshold to indicate conditional presence of the pathogen and concentration of the pathogen, (v) operating the micropump to thereby force a third fluid hosting a pathogen-selective photoluminescent label from the source to the destination, (vi) activating a light excitation subsystem, (vii) capturing one or more images from the target by an image capture subsystem, (viii) analyzing the one or more captured images to determine presence of the pathogen-selective photoluminescent label thus double-confirming a) presence of the pathogen, and b) concentration of the pathogen, (ix) obtaining a second signal from the mass measurement subsystem indicating a second mass change between steps (v) and (vi), and (x) analyzing the second mass change to determine presence of the pathogen-selective photoluminescent label thus triple-confirming a) presence of the pathogen, and b) concentration of the pathogen.

In the above method, the mass measurement subsystem includes a quartz crystal microbalance (QCM) adapted to provide a signal corresponding to mass change thereon based on a shift in resonant frequency of the QCM.

In the above method, the selective pathogen is Salmonella Typhimurium, the pathogen-selective antibody is Anti-Salmonella CSA-1, and the pathogen-selective photoluminescent label is FITC-labeled anti-Salmonella CSA-1.

In the above method, the light excitation subsystem is adapted to project light in the range from 250 nm to 900 nm.

In the above method, the pathogen-selective photoluminescent label produces a fluorescence signal.

In the above method, the pathogen-selective photoluminescent label produces a phosphorescence signal.

In the above method, the pathogen-selective photoluminescent label is a small-molecule organic fluorophore.

In the above method, the pathogen-selective photoluminescent label is a nano-particle.

In the above method, the nano-particle is one of a semiconductor-type quantum dot or an up-converting nanoparticle.

In the above method, the image capture subsystem is a smartphone.

In the above method, the image capture subsystem includes a complementary metal oxide semiconductor sensor or a charge-coupled device or a charge-coupled device adapted to capture photoluminescence images.

In the above method, the processing subsystem includes a processor operating the smartphone.

In the above method, volume of the second fluid forced by the micropump is based on a predetermined schedule depending on the pathogen.

### BRIEF DESCRIPTION OF FIGURES

FIG. 1 is a simple schematic of a Quartz Crystal Microbalance (QCM) system to illustrate the major components thereof.

FIG. 2 is graph of admittance (1/impedance) vs. frequency, showing a shift of natural frequency due to change in mass measured by measuring a change in admittance.

FIG. 3 is a schematic which demonstrates application of nanoparticles in connection with a QCM system.

FIG. 4 is a schematic showing steps involved in testing for presence of a pathogen using the system of the present disclosure.

FIG. 5 provides chemical structures involved in the steps shown in FIG. 4.

FIG. 6A is a partial perspective view of one of the modules of the system of the present disclosure.

FIG. 6B is another partial perspective view of another of the modules of the system of the present disclosure.

FIG. 6C is a perspective view of yet another of the modules of the system of the present disclosure including a flowcell but also including other modules.

FIG. 7 is an operational schematic of Module 1 shown in FIG. 6A.

FIG. 8 is a schematic of the flowcell of FIG. 6C.

FIG. 9 is a schematic of the flowcell of FIG. 8 over the quartz crystal providing the orientation of the inlet and outlet of the flowcell over the quartz crystal.

FIG. 10 is a graph of frequency vs. time, where the Y-axis represents the resonant frequency of the 5 MHz quartz crystal showing how as the crystal interacts with each material, the natural frequency changes, indicating a reaction between the QCM surface and the molecules of each material.

FIG. 11 is a graph of frequency shift vs. time, where a close look at frequency changes is provided to clearly show the effect of the different concentrations of a pathogen (Salmonella Typhimurium) in terms of frequency shift.

FIGS. 12A, 12B, 12C, 12D, 12E, 12F, 12G, and 12H are photographs showing Fluorescein isothiocyanate (FITC) beads that were dropped on the gold surface of the quartz crystal at different concentrations to show the viability of fluorescence imaging system of the present disclosure.

FIGS. 13A, 13B, 13C, and 13D are photographs showing FITC-labeled anti-Salmonella CSA-1 antibodies bound to Salmonella Typhimurium.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel approach is described herein to quickly test for biological pathogens using a portable system. Towards this end, a dual- and triple-modality Quartz Crystal Microbalance (QCM) system combining a smartphone, an in-situ optical imaging subsystem, and a flow injection component is disclosed. This system enables a smartphone to receive real-time frequency data via Bluetooth, while a camera detects the presence of bacteria on the quartz crystal surface of the QCM using a pathogen-selective photoluminescent label. The imaging subsystem utilizes a camera to capture the bacteria optical signal, while the flow injection subsystem employs a mini peristaltic pump and controller to introduce biochemical solutions, antibodies, and bacteria. All components are contained package that is portable. According to one embodiment, Fluorescein isothiocyanate (FITC) images were captured with 5 MHz quartz crystals when a prototype system was tested.

The present disclosure provides a smartphone-based biosensor for detecting a variety of pathogens, e.g., Salmonella Typhimurium, using a combination of a small flow injection system and an imaging system integrated with a QCM circuit. The biosensor system of the present disclosure is adapted for dual- and triple-modalities of near-simultaneous detection of mass change and optical imaging, e.g., fluorescent imaging, providing a comprehensive approach to pathogen detection. The QCM circuit, equipped with a Bluetooth module, sends frequency and temperature data to the smartphone for real-time monitoring. The biosensor also includes an LED-based optical excitation source and a peristaltic pump which are designed to work together. The flow injection system introduces solutions containing pathogen-specific antibodies, pathogen-selective photoluminescent label, and solutions that are potentially containing the pathogens to the surface of the quartz crystal. The frequency changes are measured throughout the entire process, from immobilizing antibodies to detecting the pathogen. At the end of the process, photoluminescence images including fluorescence images are captured using the smartphone's camera to confirm the pathogen's presence. This integration of QCM, flow injection system, fluorescence imaging, and smartphone technology allows for rapid and on-site detection of pathogens with the convenience and portability of a smartphone-based biosensor.

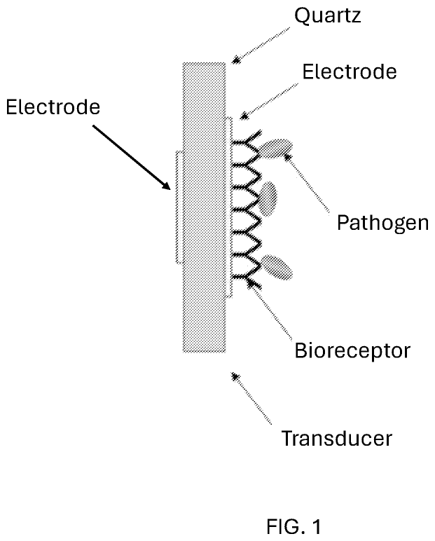

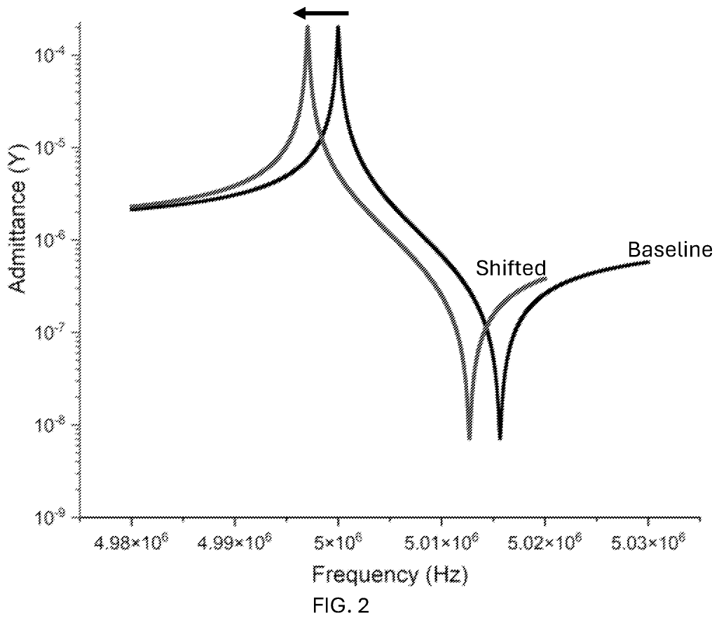

A QCM is a mass-based biosensor that quantifies a change in mass resulting from bio-molecular interactions occurring on the quartz crystal surface. It comprises a quartz crystal, a bioreceptor, and a transducer to record frequency signals. Referring to FIG. 1, a simple schematic of a QCM is provided to illustrate the major components of the QCM. The QCM sensor's functioning can be categorized into three main stages: bioreceptor immobilization techniques, antibody-antigen reaction, and signal acquisition. Initially, the driving circuit stimulates the quartz crystal, causing it to oscillate at a precise resonant frequency. Subsequently, biological recognition molecules—such as antibodies, aptamers, or engineered protein scaffolds—are attached to the crystal's surface to capture specific targets. Finally, the antibodies capture target analytes, leading to real-time measurements of changes in frequency. For this process to occur, the quartz crystal vibrates at a resonant frequency when a voltage is applied across the two electrodes, shown in FIG. 1. A baseline for natural frequency is first established of the mass with the biological recognition molecules that bind to one of the two electrodes. Once the pathogen is captured by the biological recognition molecules, the mass changes slightly, thus shifting the natural frequency towards lower frequencies. This shift is shown in FIG. 2 which is a graph of admittance (1/impedance) vs. frequency showing the shift of natural frequency due to change in mass measured by measuring a change in admittance.

Nowadays, nanoparticles are crucial in analyzing target analytes, enhancing both the quantification and sensitivity of biosensors. This increased sensitivity is vital in food safety, as even trace amounts of pathogens can cause severe issues. Extensive research on biologically-functionalized nanoparticles has been conducted to improve the performance of both biological recognition molecules and transducers. Nanoparticles exhibit excellent biocompatibility, unique optical properties, and a large specific surface area. Nanoparticles have accelerated the detection of viruses and bacteria in the field of food safety, where they serve as both labels and signal amplifiers in immunoassays. These nanoparticles come in various types depending on their adjustable size and shape, including nanorods, nanowires, nanotubes, and nanobelts, all of which are employed in biosensor platforms. Among the most widely used metal nanoparticles is AuNPs. AuNPs, in conjunction with QCM sensors, have been used to enhance sensitivity. This method demonstrated the capability to detect E. coli O157:H7 at concentrations five orders of magnitude (typically measures in colony forming unit (CFU) per mL) lower than standard technique thus demonstrating marked improvement over these standard techniques.

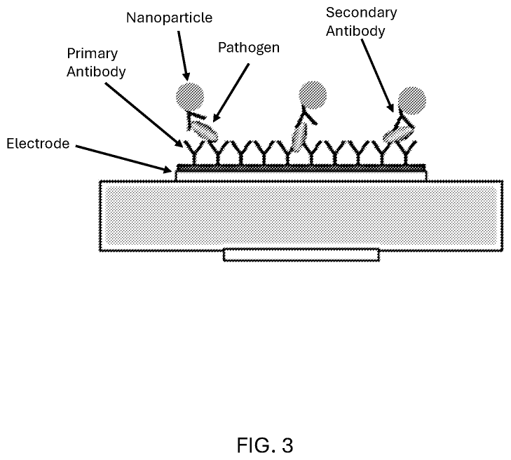

Referring to FIG. 3, a schematic is shown which demonstrates application of such nanoparticles. As shown in FIG. 3, a primary pathogen specific antibody, aptamer or other biological recognition system binds to the surface of the electrode and is capable of attracting and binding to the pathogen. Next, a secondary pathogen-specific antibody or other biological recognition molecule with nanoparticles binds to the pathogen. Nanomaterials exhibit unique optical, electrical, or catalytic properties. Among these nanomaterials, gold nanoparticles (AuNPs) stand out as particularly attractive due to their diverse properties, which include vibrant coloration, high biocompatibility, a large surface-to-volume ratio, low toxicity, and catalytic activity. These properties make AuNPs advantageous in enhancing signals in various aspects of biosensors, such as colorimetric sensing, fluorescence-based sensors, as well as QCM sensors.

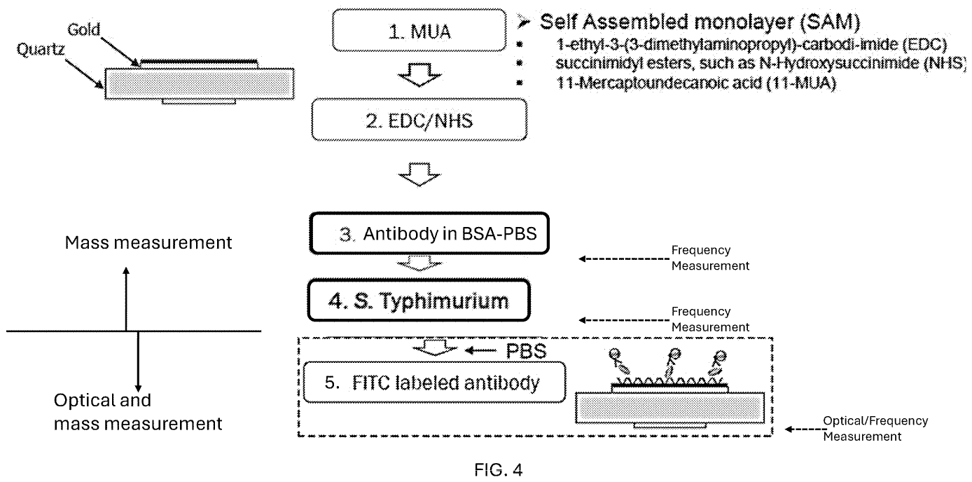

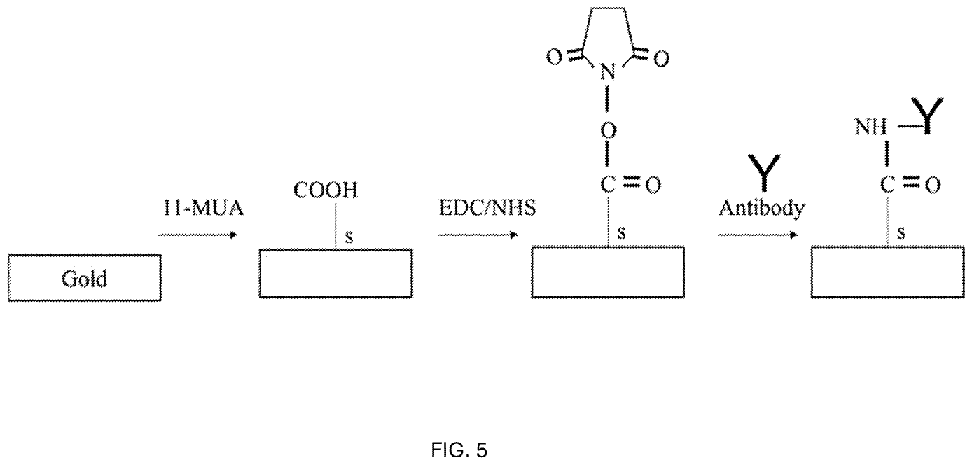

To demonstrate the process of the present disclosure, reference is made to FIG. 4, which is a schematic showing the steps involved in testing for the presence of a pathogen. These steps were taken as parts of two separate experiments but combined here as one experiment description. The chemical structures of each step are shown in FIG. 5. A 5 MHz crystal (AT5-14-12-AU-WRAP, openQCM, Italy) was used which has gold electrodes on the top surface of the quartz crystal surface. Each chemical was transferred to the gold surface using a micropump (a peristaltic pump). First, 500 μL of 11-Mercaptoundecanoic acid (11-MUA) was channeled to the quartz crystal to create thiol groups on the gold surface, producing a monolayer of MUA with COOH groups. Once this 11-MUA was transferred to the gold surface, the pump was stopped, and the reaction was allowed to proceed for 1 hour. Next, 250 μL of 1-ethyl-3-(3-dimethylaminopropyl) carbodiimide hydrochloride (EDC) and 250 μL of N-hydroxysuccinimide (NHS) were mixed, injected, and allowed to react for 1 hour to form NHS esters. These active NHS esters target primary amines on the antibody. Next, 500 μL of antibodies specific to a pathogen, e.g., Salmonella Typhimurium were prepared using 1% BSA-PBS (a 0.01 M phosphate-buffered saline solution (PBS) containing 0.138 M NaCl and 0.0027 M KCl at pH 7.4, injected and the pump was stopped for 1 hour, resulting in their immobilization on the surface. The antibody concentration was 200 μg/mL. In the case of Salmonella Typhimurium the Anti-Salmonella CSA-1 antibody, as described below the FITC-labeled anti-Salmonella CSA-1 antibody are commercially available. To test the efficacy of the system, after antibody immobilization, 1 mL of Salmonella Typhimurium was injected, and they were captured by the immobilized antibodies. PBS was injected to rinse and eliminate any non-specific binding between each step, while all frequencies were measured in real-time. The experiments were conducted for four different concentrations ranging from 10³ CFU/mL to 10⁹ CFU/mL, with a new quartz crystal used for each set. Three repetitions were performed for the calibration data set, and a single experiment was conducted as a control using PBS without Salmonella Typhimurium, as well as for specificity testing. The change in mass resulting from the binding of the Salmonella Typhimurium to the Anti-Salmonella CSA-1 antibody results in a shift in natural frequency of the quartz crystal. This change in natural frequency indicates i) presence of the pathogen, and ii) concentration of the pathogen. Finally, FITC-labeled anti-Salmonella CSA-1 was injected for binding to the Salmonella Typhimurium for optical and mass measurements. Again PBS was injected to rinse and eliminate any non-specific binding. The FITC-labeled anti-Salmonella CSA-1 allows for optical measurements which requires a light source and an image capture device. The optical measurements is used to double-confirm the results of the mass measurements as far as i) presence of the pathogen and ii) concentration of the pathogen based on the optical measurements and subsequent analysis. Finally, the mass measurement approach can be used again after the binding of the FITC-labeled anti-Salmonella CSA-1 to Salmonella Typhimurium, by measuring another shift in natural frequency, to triple-confirm i) presence of the pathogen and ii) concentration of the pathogen.

To achieve a portable version of the system according to the present disclosure a smartphone-based QCM system was developed. A smartphone (SAMSUNG S22 Ultra) was chosen owing to its advanced features, including a high-quality camera, manual focus control, and adjustable exposure times. The smartphone was combined with the QCM system and fluorescence imaging system. The smartphone plays a pivotal role in the system, serving both as a data acquisition tool and an imaging device. Its primary function is to receive, display and analyze the real-time resonant frequency and temperature data from the QCM device via a Bluetooth module. The other function is to capture images through the camera before and after pathogen-selective photoluminescent label (e.g., FITC-labeled anti-Salmonella CSA-1) binding to the pathogen (e.g., Salmonella Typhimurium).

The system also includes a Pierce oscillator, known to a person having ordinary skill in the art, for its frequency stability and minimal components. This quartz crystal oscillator was incorporated into the openQCM Teensy shield (openQCM-TEENSY-2 from openQCM, Italy) which was specifically designed to be compatible with a Teensy 3.2 microcontroller (PJRC, USA). The Teensy microcontroller, equipped with openQCM firmware, demonstrates versatility, allowing precise frequency measurements ranging from 1 kHz up to 65 MHz while monitoring the temperature as well. The firmware was modified to integrate the shield, with the inclusion of a Bluetooth module for enhanced connectivity in this study. This integrated shield offers essential features, including a 3V3 voltage regulator, a signal conditioning circuit, an Inter-Integrated Circuit (I2C) temperature sensor, and a crystal oscillator driver (SN74LVC1GX04) that generates a square wave output. Establishing a physical connection with a flowcell (O100FC, Stanford Research Systems, USA), is facilitated through a USB connector.

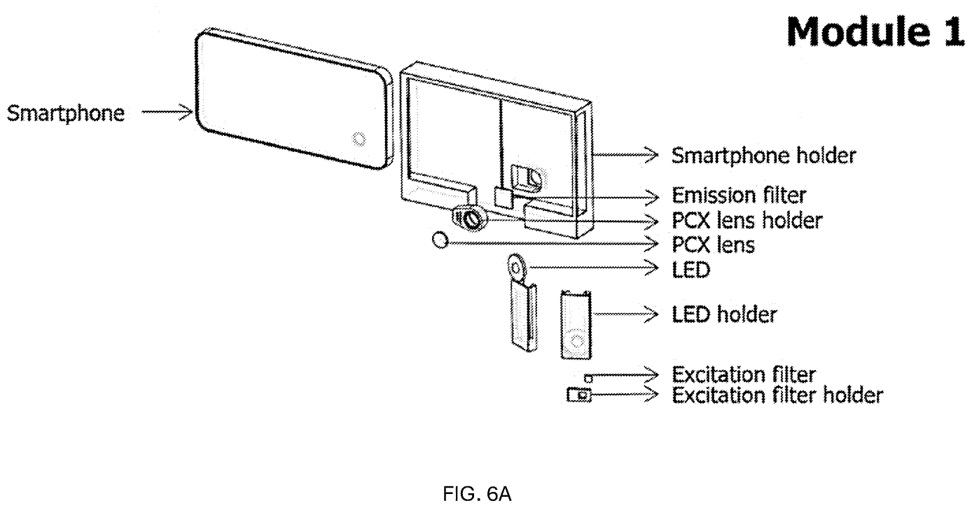

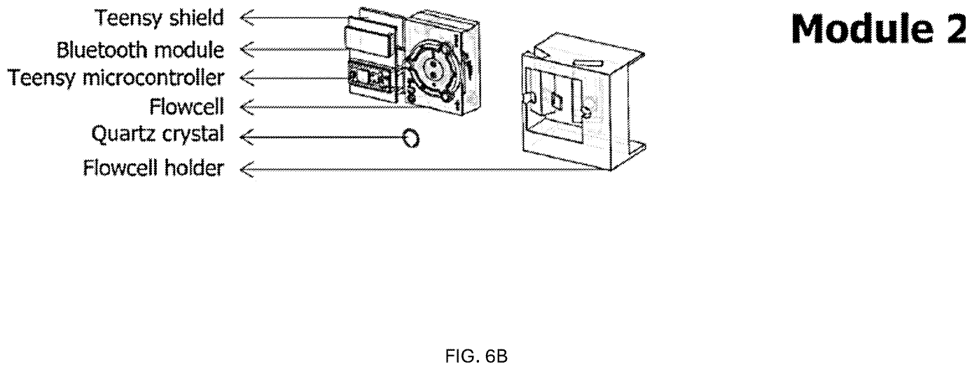

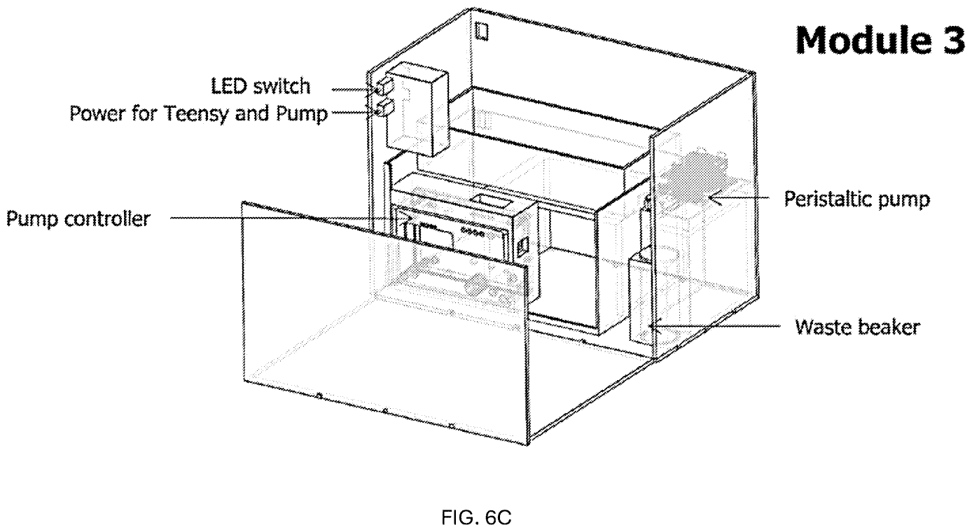

Altogether, 3 modules comprised the system: fluorescence imaging (Module 1), electronics (Module 2), and the microfluidics system (Module 3). Each element resides within a casing, with each casing designed to fulfill specific functions related to the optical, circuit, and flow injection components. Referring to FIGS. 6A, 6B, and 6C all three modules are shown in partial exploded perspective views.

Module 1 primarily focuses on the fluorescence imaging system. A smartphone holder was purposefully designed to securely hold a smartphone in a horizontal orientation. The smartphone holder incorporates the smartphone, a plano-convex lens (PCX) (#37-783, Edmund, USA), enclosed in an optical holder (LMR05, Thorlabs, USA), and an emission filter (2020OFS-525 Bin 4.G.8, Optical filter shop, USA), aligned to optimize optical performance. Two excitation filters (2020° F.S-480 Bin 4.H.8, Optical Filter Shop, USA) were enclosed in their respective holders, and two 490 nm LEDs (Fedy, China) were also secured with the holders. The LEDs and excitation filters form an integral part of the flowcell holder in Module 2 and are positioned at an angle of 24 degrees in front of the quartz crystal.

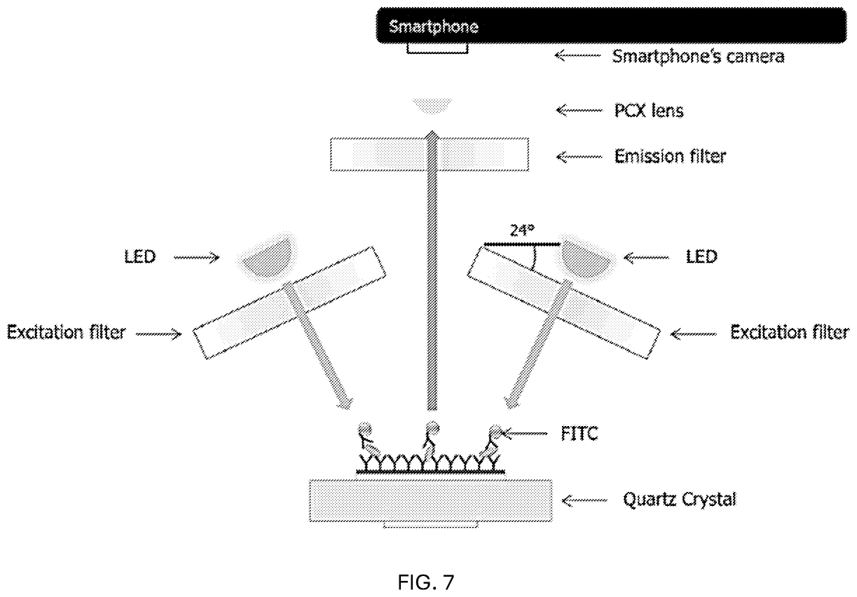

Module 1 is adapted to capture the emission light from Salmonella Typhimurium reacted to the FITC-labeled antibody on the quartz crystal enclosed in the flowcell. Two 490 nm LEDs with excitation filters excite the FITC-labeled antibody, while the smartphone's camera acquires the intensities from Salmonella Typhimurium cells reacted to the FITC-labeled antibody through the emission filter as shown in FIG. 7, which is an operational schematic of Module 1. The excitation filters and 490 nm LEDs were tilted by 24 degrees based on the smartphone's positioning. This angle was optimized to obtain the highest intensity from the FITC-labeled antibody at a specific angle and minimize the distance between the smartphone and the quartz crystal, based on the focal length of the PCX lens.

Module 2 represents the primary oscillation circuit system. It features a flowcell holder designed to house LEDs, excitation filters, and a flowcell. The flowcell connects to the Teensy shield via a USB connection, which interfaces with both a Bluetooth module and a Teensy microcontroller. The quartz crystal will be positioned on the flowcell and then inserted into the flowcell holder.

Module 2 is adapted for maintaining quartz crystal oscillation and facilitating resonant frequency measurements through smartphone connectivity. Module 2 includes Circuit Boards 1 and 2. Circuit Board 1 was combined with Circuit Board 2 to incorporate the Teensy 3.2 microcontroller and a Bluetooth module HC-05 (AMAZON, USA). The Bluetooth module ensures communication and data exchange with the smartphone. Circuit Board 1 and Circuit Board 2 were designed together to optimize space utilization and functionality. Since the Teensy 3.2 microcontroller with pre-installed openQCM firmware, the code was modified to integrate the Bluetooth HC-05 module. Consequently, this enabled the smartphone to obtain real-time frequency and temperature data via the Bluetooth interface. Circuit Board 3 which is in Module 3, includes power management circuits, an LED driver, LEDs, capacitors, a voltage regulator, a heat sink, two switches, and power sources for both the microcontroller and the peristaltic pump. The power is linked to a power connector, allowing connection to a 12V AC adapter that draws power from the wall. Capacitors and a voltage regulator are employed to stabilize the voltage at 5V. This stable power is then distributed to the peristaltic pump, LEDs, and the Teensy microcontroller.

Module 3 encompasses the entire system, inclusive of a microfluidic system. The housing for the peristaltic pump, located under the peristaltic pump, is designed to align with the outlet port of the flowcell at a corresponding level. Additionally, an enclosure of a waste beaker was made, reducing the risk of contamination. A switch holder accommodates two switches, one for the LEDs and the other for the power source. The power source switch is used to power both the pump and microcontroller, and it's connected to a port for plugging in an adapter, allowing the conversion of wall power into a 12-volt AC supply.

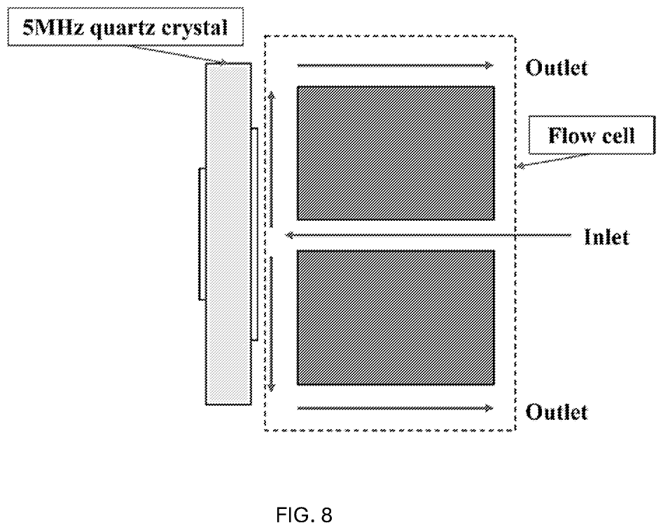

Module 3 serves as a pivotal component of the microfluidic system, facilitating the introduction of liquid samples onto the quartz crystal at a consistent flow rate. The microfluidic system includes the pump controller, the mini peristaltic pump, and a waste beaker. A pump controller (CECS-0100, TAKASAGO FLUIDIC SYSTEMS, Japan) accommodates a wide range of lower flow rates spanning from 0.23 to 350 μl/min. The controller was manually adjusted, and the rate was fixed for the entire experiment. A versatile 6-channel peristaltic pump (RP-6R01S-3P6A-DC10VS, TAKASAGO FLUIDIC SYSTEMS, Japan) is positioned near the outlet port to minimize any potential disruptions caused by its operation. The pump was linked to the openQCM sensor module (openQCM-SENS-MOD-03, openQCM, Italy) via tubes that housed the quartz crystal. This sensor module is also referred to herein as the flowcell. A schematic of the flowcell is shown in FIG. 8. This sensor module includes a transparent fluidic cover, an O-ring, a temperature sensor, and a USB connection. The flowcell is strategically oriented in a vertical configuration to minimize the risk of air bubbles interfering with measurements, with liquid introduced from the bottom. Importantly, the inlet port is positioned at a lower elevation than the outlet port, ensuring a smooth flow trajectory.

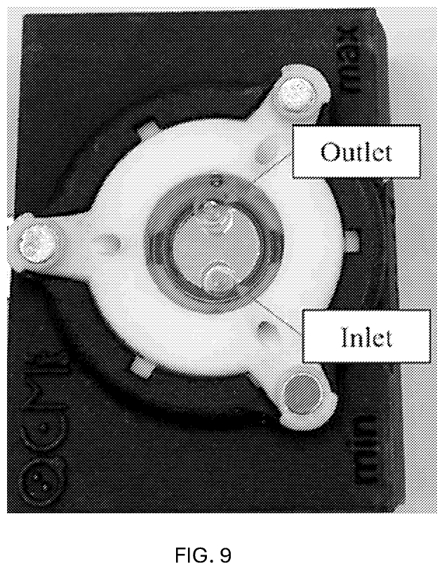

Referring to FIG. 9, a schematic of the flowcell over the quartz crystal is shown providing the orientation of the inlet and outlet of the flowcell over the quartz crystal. Once the quartz crystal is correctly positioned, the flowcell is sealed by the cover, which includes inlet and outlet tube holes. Next, the tubes are inserted into those holes to allow for ingress and egress of fluid.

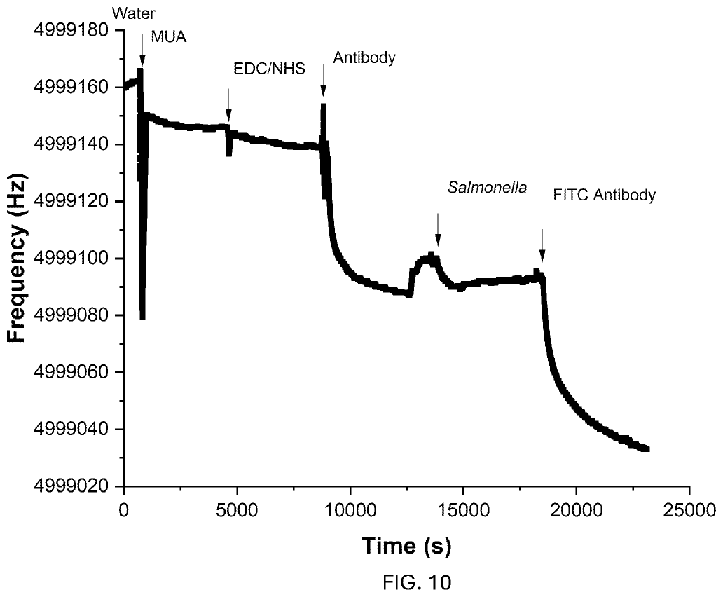

The experiment's frequency shifts are illustrated in FIG. 10, which is a graph of frequency vs. time, where the Y-axis represents the resonant frequency of the 5 MHz quartz crystal. As the crystal interacted with each material, the frequency changed, indicating a reaction between the QCM surface and the molecules. The frequency shift observed before and after the injection of 10⁹ CFU/mL of Salmonella Typhimurium was-6.7 Hz with a standard deviation of 0.69 Hz. Similarly, the frequency shift observed before and after the FITC-labeled antibody was −59.7 Hz with a standard deviation of 0.2 Hz. The combined frequency shift was about −66.4 Hz, indicating that the presence of Salmonella Typhimurium caused the frequency shift, which was improved by the FITC-labeled anti-Salmonella CSA-1 antibodies. In FIG. 10, the arrows represent the starting point when the solution was injected.

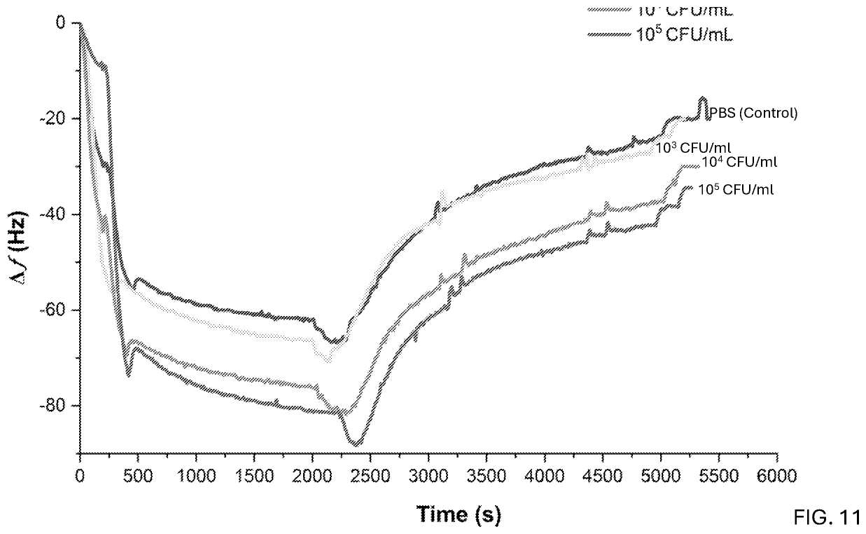

Referring to FIG. 11, which is a graph of frequency shift vs. time, a close look at frequency changes is provided to clearly show the effect of the different concentrations of Salmonella Typhimurium. This figure presents frequency recordings after the FITC-labeled antibody and the subsequent washing steps. S. Typhimurium concentrations of 10³, 10⁴, and 10⁵ CFU/mL, which are close to the limit of detection (LoD) of 5 MHz, were tested using a 10 MHz quartz crystal. Compared to 5 MHz, the frequency rapidly decreased after injecting the FITC-labeled antibody and increased after PBS due to higher sensitivity. The most pronounced frequency drop was observed at a concentration of 10⁵ CFU/mL, where the frequency drop reached approximately-84 Hz which was the minimum point. In comparison, for a 5 MHz quartz crystal, the same concentration resulted in a frequency drop of approximately-29 Hz based on Table 1. This observation suggests that the 10 MHz quartz crystal exhibits higher frequency shifts than the 5 MHz quartz crystal for the same concentration, highlighting the superior sensitivity of the 10 MHz quartz crystal.

TABLE 1
Frequency shifts for S. Typhimurium concentrations
at 5 MHz. Data points represent the average frequency.

| | Average minimum | | Average frequency shifts | |
|---|---|---|---|---|
| Plate counting | frequency point (Hz) | | before and after FITC (Hz) | |
| methods (CFU/mL) | Δ f | SD | Δ f | SD |
| 0 | −24.8 | | −11 | |
| 10⁵ | −29 | 4.9 | −7.5 | 0 |
| 10⁷ | −66.25 | 5.3 | −54.5 | 0.707 |
| 10⁹ | −103.5 | 6.5 | −86 | 0.707 |

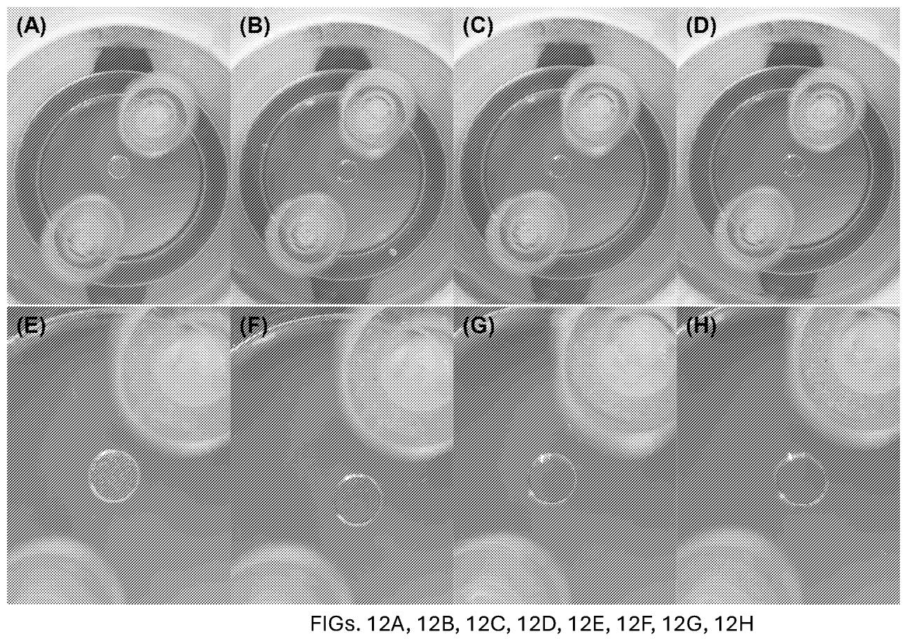

Referring to FIGS. 12A-12H, which provide images captured by the smartphone, displays 4 μm FITC beads that were dropped on the gold surface of the quartz crystal to test the fluorescence imaging system. The concentrations used were 1 mg/mL (FIG. 12A), 0.1 mg/mL (FIG. 12B), 0.01 mg/mL (FIG. 12C), and 0.001 mg/mL (FIG. 12D). FIGS. 12E, 12F, 12G, and 12H are the magnified images of FIGS. 12A, 12B, 12C, and 12D, respectively, show that higher concentrations resulted in higher intensities of the fluorescence dots.

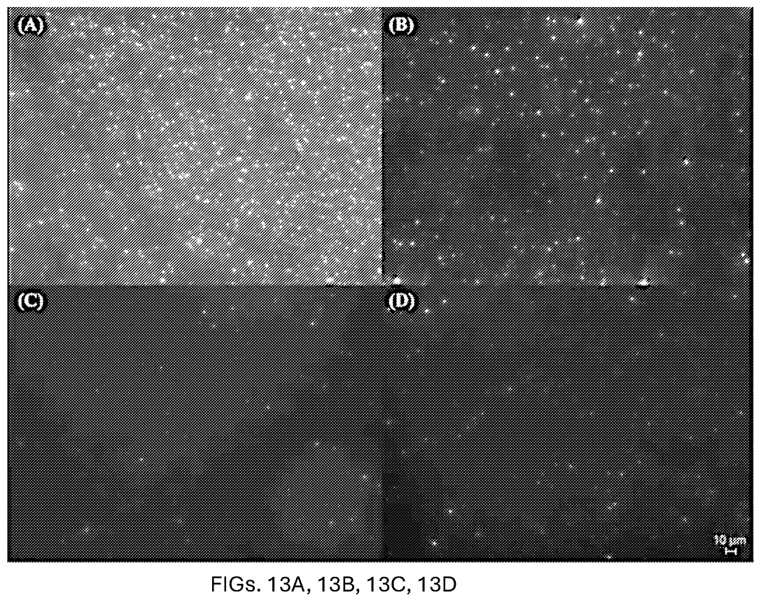

To visually confirm the presence of captured Salmonella Typhimurium on the quartz crystal surface, fluorescence images were captured using a Keyence microscope (BZ-X800 Series, USA). Referring to FIGS. 13A, 13B, 13C, and 13D photographs of Salmonella Typhimurium labeled with the FITC-labeled anti-Salmonella CSA-1 antibodies at different concentrations, representing 10⁹ CFU/mL in FIG. 13A, 10⁷ CFU/mL in FIG. 13B, 10⁵ CFU/mL in FIG. 13C and 0 CFU/mL in FIG. 13D on the 5 MHz quartz crystal surface. These images serve as crucial visual evidence of Salmonella Typhimurium capture. By analyzing these images, one can double-confirm i) presence of the Salmonella Typhimurium, and ii) concentration of Salmonella Typhimurium as compared to mass measurement alone.

Presence and concentration of Salmonella Typhimurium on cantaloupe rinds was also investigated to determine the average minimum frequency point, and the average frequency shifts before and after the FITC-labeled antibody. Table 2 provides the results of the frequency and frequency shifts.

TABLE 2
Frequency shift for Salmonella Typhimurium detection
on cantaloupe rinds at 10 MHz QCM crystal

| Plate counting | Minimum frequency | Average frequency shifts |
|---|---|---|
| methods (CFU/mL) | point (Hz) | before and after FITC (Hz) |
| 0 | −21.0 | 2.78 |
| 4.25 × 10⁵ | −31.0 | −10.78 |
| CFU/mL | −71.0 | −25.66 |
| | −77.5 | −25.86 |

Those having ordinary skill in the art will recognize that numerous modifications can be made to the specific implementations described above. The implementations should not be limited to the particular limitations described. Other implementations may be possible.

1. A pathogen detection system, comprising:
a microfluidic channel adapted to transfer a volume of fluid from a source to a destination, the destination defining a target;
a micropump coupled to the microfluidic channel and configured to force fluid along the microfluidic channel from the source to the destination;
a mass measurement subsystem adapted to measure changes in mass of a substance at the destination of at least 1 nanogram;
a light excitation subsystem adapted to project light onto the target;
an image capture subsystem adapted to monitor the target and obtain images therefrom; and
a processing subsystem executing instructions held in a non-transitory memory, the processing subsystem adapted to:
(i) operate the micropump to thereby force a first fluid hosting a pathogen-selective antibody from the source to the destination,
(ii) operate the micropump to thereby force a second fluid potentially hosting the selective pathogen from the source to the destination,
(iii) obtain a signal from the mass measurement subsystem indicating a mass change between steps (i) and (ii),
(iv) compare the mass change to a predetermined threshold to indicate conditional presence of the pathogen and concentration of the pathogen,
(v) operate the micropump to thereby force a third fluid hosting a pathogen-selective photoluminescent label from the source to the destination,
(vi) activate the light excitation subsystem,
(vii) capture one or more images from the target by the image capture subsystem, and
(viii) analyze the captured one or more images to determine presence of the pathogen-selective photoluminescent label thus double-confirming a) presence of the pathogen, and b) concentration of the pathogen.

2. The system of claim 1, wherein the mass measurement subsystem includes a quartz crystal microbalance (QCM) adapted to provide a signal corresponding to mass change thereon based on a shift in resonant frequency of the QCM.

3. The system of claim 1, wherein the selective pathogen is Salmonella Typhimurium, the pathogen-selective antibody is Anti-Salmonella CSA-1, and the pathogen-selective photoluminescent label is FITC-labeled anti-Salmonella CSA-1.

4. The system of claim 1, wherein the light excitation subsystem is adapted to project light in the range from 250 nm to 900 nm.

5. The system of claim 1, wherein the pathogen-selective photoluminescent label produces at least one of a fluorescence signal and a phosphorescence signal.

6. (canceled)

7. The system of claim 1, wherein the pathogen-selective photoluminescent label is at least one of a small-molecule organic fluorophore and a nano-particle.

8. (canceled)

9. The system of claim 7, wherein the nano-particle is one of a semiconductor-type quantum dot or an up-converting nanoparticle.

10. (canceled)

11. The system of claim 1, wherein the image capture subsystem includes a complementary metal oxide semiconductor sensor or a charge-coupled device or a charge-coupled device adapted to capture photoluminescence images.

12. The system of claim 1, wherein the processing subsystem includes a processor operating the smartphone.

13. The system of claim 1, wherein volume of the second fluid forced by the micropump is based on a predetermined schedule depending on the selected pathogen.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. A method of detecting a pathogen, comprising:
(i) operating a micropump to thereby force a first fluid hosting a pathogen-selective antibody from a source to a mass measurement subsystem (destination), defining a target, via a microfluidic channel;
(ii) operating the micropump to thereby force a second fluid potentially hosting the selective pathogen from the source to the destination;
(iii) obtaining a signal from the mass measurement subsystem indicating a mass change between steps (i) and (ii);
(iv) comparing the mass change to a predetermined threshold to indicate conditional presence of the pathogen and concentration of the pathogen;
(v) operating the micropump to thereby force a third fluid hosting a pathogen-selective photoluminescent label from the source to the destination;
(vi) activating a light excitation subsystem;
(vii) capturing one or more images from the target by an image capture subsystem; and
(viii) analyzing the one or more captured images to determine presence of the pathogen-selective photoluminescent label thus double-confirming a) presence of the pathogen, and b) concentration of the pathogen.

28. The method of claim 27, wherein the mass measurement subsystem includes a quartz crystal microbalance (QCM) adapted to provide a signal corresponding to mass change thereon based on a shift in resonant frequency of the QCM.

29. The method of claim 27, wherein the selective pathogen is Salmonella Typhimurium, the pathogen-selective antibody is Anti-Salmonella CSA-1, and the pathogen-selective photoluminescent label is FITC-labeled anti-Salmonella CSA-1.

30. The method of claim 27, wherein the light excitation subsystem is adapted to project light in the range from 250 nm to 900 nm.

31. The method of claim 27, wherein the pathogen-selective photoluminescent label produces at least one of a fluorescence signal and a phosphorescence signal.

32. (canceled)

33. The method of claim 27, wherein the pathogen-selective photoluminescent label is at least one of a small-molecule organic fluorophore and a nano-particle.

34. (canceled)

35. The method of claim 33, wherein the nano-particle is one of a semiconductor-type quantum dot or an up-converting nanoparticle.

36. The method of claim 27, wherein the image capture subsystem is a smartphone.

37. The method of claim 27, wherein the image capture subsystem includes a complementary metal oxide semiconductor sensor or a charge-coupled device or a charge-coupled device adapted to capture photoluminescence images.

38. (canceled)

39. The method of claim 27, wherein volume of the second fluid forced by the micropump is based on a predetermined schedule depending on the pathogen.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)
